(19) United States
(12) Patent Application Publication
(10) Pub. No.: US 2018/0044640 A1
(43) Pub. Date: Feb. 15, 2018

(54) CONTRACTILE CELLULAR CONSTRUCT FOR CELL CULTURE

(71) Applicant: Agency for Science, Technology and Research, Singapore (SG)

(72) Inventors: Hong Fang Lu, Singapore (SG); Meng Fatt Leong, Singapore (SG); Tze Chiun Lim, Singapore (SG); Andrew Chwee Aun WAN, Wan, Singapore (SG)

(21) Appl. No.: 15/553,285

(22) PCT Filed: May 16, 2016
(86) PCT No.: PCT/SG2016/050228

§ 371 Date: Aug. 24, 2017

(30) Foreign Application Priority Data
May 15, 2015 (SG) 10201503869P

(51) Int. Cl. C12N 5/077; G01N 33/50; G01N 33/483

(52) U.S. Cl. CPC C12N 5/0657; G01N 33/4833; G01N 33/5061; G01N 33/5082; C12N 2506/45; C12N 2533/30; C12N 2500/90; C12N 2513/00

(57) ABSTRACT

The present invention relates to a method for producing a contractile cellular construct, comprising the steps of: a) seeding pre-selected cells onto a mold, wherein the pre-selected cells comprise signal emitting agents; and b) culturing the pre-selected cells to produce the cellular construct. The said method is exemplified by mixing cardiomyocytes with fluorescent microbeads, seeding the suspension on a polydimethylsiloxane (PDMS) mold and culturing to form a contractile device comprising of cardiac muscles. The present invention also relates to a method for measuring the contractility of the cellular construct by measuring the displacement of the signal emitting agents, and a method for screening a method for screening one or more agents for modulating the contractility of a contractile cellular construct.

### FIELD OF THE INVENTION

The present invention relates to a method of producing a contractile cellular construct. In particular, the present invention relates to a method of producing a 3D contractile cellular construct for cell culture.

### BACKGROUND OF THE INVENTION

Pluripotent stem cells provide an unlimited ex vivo source of differentiated cells which provide unique opportunities to model disease, to establish personal predictive drug toxicology and target validation, ultimately to enable autologous cell-based therapies. An example of a differentiated cell type obtained from pluripotent stem cells are cardiomyocytes.

Mature adult cardiomyocytes in native heart are surrounded by an organization of supporting matrix and neighbouring cells with gradients of chemical, electrical and mechanical signals. Consequently, various approaches have been reported as in vitro cardiac models that better mimic the native heart tissue architecture and functions. These include 2-dimensional (2D) and 3-dimensional (3D) cell constructs formed by cells seeded and organized in either polymeric or biological scaffolds, or extracellular matrix (ECM) hydrogels such as collagen, matrigel, laminin, fibronectin, and fibrin, or decellularized natural matrix as well as the cellular tissues organized from stackable cell sheets. However, while these studies highlighted the utility of cardiac tissue constructs, the constructs containing exogenous scaffolds or ECM-based materials may interfere with direct cell-cell interaction, limiting the recapitulation of native microcellular structure. Furthermore, the use of animal cells or animal-derived extracellular matrix scaffolds poses a potential issue when considering the translational use of these constructs in human.

In addition, cardiac toxicity is a leading cause for drug attrition during the clinical development of pharmaceutical products and has resulted in numerous preventable patient deaths. Currently, several in vitro cardiac toxicity models based on human ESC or iPSC-derived cardiac cells have been used for drug tests, such as FLIPR® Tetra system and xCELLigence RTCA Cardio system. However, these in vitro toxicity screens either rely on costly, specially manufactured tissue culture plates and/or the characterization of single cardiac ion channels in cardiac cells, which do not accurately model pertinent biochemical characteristics of the human heart, thus limiting their pharmaceutical application.

There is therefore a need to provide an efficient in vitro culture system both for the production of functional differentiated cells, such as cardiomyocytes, and monitoring cell function profile that overcomes, or at least ameliorates, one or more of the disadvantages described above.

### SUMMARY

In one aspect, there is provided a method for producing a contractile cellular construct, comprising the steps of: a) seeding pre-selected cells onto a mold, wherein the pre-selected cells comprise signal emitting agents; and b) culturing the pre-selected cells to produce the contractile cellular construct.

In one aspect, there is provided a contractile cellular construct produced by the method as described herein.

In one aspect, there is provided a method of measuring the contractility of the contractile cellular construct as described herein, comprising:

a) measuring the location of the signal emitting agents in the contractile cellular construct, at two or more pre-selected times; and
b) determining the temporal change in the location of the signal emitting agents to produce a contraction profile and relaxation profile based upon the measurements of a).

In one aspect, there is provided a method for screening one or more agents for modulating the contractility of a contractile cellular construct, comprising:

a) contacting the contractile cellular construct as described herein with said one or more agents;
b) measuring the location of the signal emitting agents, comprised in the contractile cellular construct, at two or more pre-selected times;
c) determining the temporal change in the location of the signal emitting agents in said two or more pre-selected times, to produce a test contraction profile and test relaxation profile based upon the measurements of b);
d) comparing the test contraction profile and test relaxation profile of c) with a control contraction profile and control relaxation profile of a contractile cellular construct as described herein that has not been contacted with the one or more agents or has been contacted with the one or more agents at a different concentration to that of step a); wherein a differential profile between the test and control contraction or relaxation profiles demonstrates a modulating activity of said one or more agents on the contractile cellular construct.

### BRIEF DESCRIPTION OF THE DRAWINGS

The invention will be better understood with reference to the detailed description when considered in conjunction with the non-limiting examples and the accompanying drawings, in which:

FIG. 1 is a schematic illustration of 3D cardiac tissue fabrication.

FIG. 2 shows the differentiation of hiPSCs into cardiac cells under defined culture condition. (A) Schematic diagram of the reprogramming protocol used. (B) Black-white images of cells during differentiation. (C) RT-PCR analysis indicates the differentiated cells expressed cardiac markers. NC: negative control.

FIG. 3 shows the characterization of differentiated cardiac cells. (A) Immunofluorescence staining of cardiac markers (cTnT, NKX2.5, MYH6) in differentiated cells. Cells were counterstained for nuclei with DAPI. (B) FACS analysis of cardiac marker cTnT expressed in differentiated cells.

FIG. 4 shows 3D cardiac tissue formation. (A) Black-white images of the differentiated cardiac cells loaded into PDMS microchannels at 0, 2, 20 and48 hrs and Photo of 3D cardiac tissues. (B) Live/dead staining images of cells in 3D tissue at day 1 and day 21.

FIG. 5 shows the characterization of 3D cardiac tissue. (A) Hematoxylin/eosin staining of tissue sections. (B) Immunofluorescence staining of extracellular matrix proteins in cardiac tissue sections. Cells were counterstained for nuclei with DAPI.

FIG. 6 shows the characterization of 3D cardiac tissue. Immunofluorescence staining of cardiac markers (MYH6, cTnT, NKX2.5 and Actinin) in tissue sections. Cells were counterstained for nuclei with DAPI.

FIG. 7 shows signal emitting agent-labelled 3D cardiac tissue. Each emitting dot (arrows) corresponds to a polystyrene microsphere.

FIG. 8 shows the analysis of erythromycin toxicity response of 3D cardiac tissues using IMARIS software. The software can identify the contraction and relaxation events based on the relative positions of the signal emitting beads during the entire tissue beating sequence. These positions were recorded, and the relevant contractility parameters were calculated from the temporal change of these positions. (A) Displacement; (B) Velocity; (C) Acceleration vector.

FIG. 9 shows a summary of drug toxicity assays using 3D cardiac tissues and IMARIS software. (A) Representative contraction peak recordings of cardiac tissue exposed to cumulatively increasing drug concentrations. (B) Drug toxicity effect on tissue beating rates.

FIG. 10 shows the toxicity study (black bar) of clinical drugs using 3D fluorescence-labelled human cardiac tissues in comparison with ATP activity (grey bar). Antibiotics: erythromycin, ampicillin, trovafloxacin; antidiabetics: rosiglitazone, troglitazone, metformin.

FIG. 11 shows the preparation of 2D cardiac model for high throughput drug screening. (A) a schematic diagram illustrating the platform for fabrication of a 2D cardiac model for drug screening test. (B) Phase contrast micrographs of cultures at various time points following cell seeding demonstrate the typical cardiomyocyte monolayers on 384-well plates. (C & D) Representative image of 2D cardiomyocyte labelled with emitting agents (fluorescence beads, white arrows) in 384-well plate (C), and synchronized beating profile analyzed by Imaris software (D).

FIG. 12 shows the pentamidine toxicity response of iPSC-derived cardiomyocytes cultured in a 384-well plate. (A) Representative contraction peak recordings of cardiomyocytes exposed to cumulatively increasing drug concentrations. (B) Representative contraction peak recordings of cardiomyocytes exposed to 1 μM, 5 μM and 10 μM of pentamidine at 4 h, 16 h, 28 h and 44 h post-treatment.

### DETAILED DESCRIPTION OF THE PRESENT INVENTION

In one aspect the present invention refers to a method for producing a contractile cellular construct. The method comprises the steps of a) seeding pre-selected cells onto a mold, wherein the pre-selected cells comprise signal emitting agents; and b) culturing the pre-selected cells to produce the contractile cellular construct.

In one embodiment, prior to step a) the method further comprises: inducing a pluripotent stem cell into a pre-determined lineage of the pre-selected cells; isolating the induced pre-selected cells; and contacting the isolated pre-selected cells with signal emitting agents to produce pre-selected cells comprising signal emitting agents.

The pluripotent stem cell may be a human induced pluripotent stem cell (hiPSC). The hiPSC may be derived from a biological sample. The biological sample may be a sample of tissue or cells. The biological sample may include but is not limited to blood, blood plasma, serum, buccal smear, amniotic fluid, prenatal tissue, sweat, nasal swab or urine, organs, tissues, fractions, and cells isolated from mammals including humans. The sample may also comprise clinical isolates that may include sections of the biological sample including tissues (for example, sectional portions of an organ or tissue).

In some embodiments, prior to step a) the method further comprises: isolating the pre-selected cells from a biological sample; and contacting the isolated pre-selected cells with signal emitting agents to produce pre-selected cells comprising signal emitting agents.

The contractile cellular construct may comprise any cells having a contractile function and may be in vitro or ex vivo. In some embodiments, the contractile cellular construct may comprise muscle cells. The muscle cells may be selected from the group consisting of skeletal muscle cells, cardiac muscle cells and smooth muscle cells.

In another embodiment the contractile cellular construct may comprise cardiac cells with contractile function. For example, a construct comprising cardiac muscle cells, a cardiomyocyte-extracellular matrix (ECM) hydrogel construct, or a cardiomyocyte-polymer/biomaterial construct. The pre-selected cells may be cardiac cells. The cardiac cells may comprise one or more mammalian cells selected from the group consisting of cardiomyocytes, endocardial cells, cardiac adrenergic cells, endothelial cells, neuromuscular cells and cardiac fibroblasts. The cardiomyocytes may comprise one or more of ventricular cardiomyocytes, atrial cardiomyocytes and nodal cardiomyocytes.

In one embodiment, the cardiac cells may comprise cardiomyocytes expressing one or more markers selected from the group consisting of MYH6, α-sarcomeric actin, cTnT, Connexin 43, GATA4, Tbx5, MEF2c, sarcomeric MHC, sarcomeric actinin, Cardiac troponin I, atrial natriuretic peptide, Smooth muscle α-actin, desmin and NKX2.5.

In some embodiments, at least 70%, 80% or 90% of the cardiac cells are cardiomyocytes.

In one embodiment, the mold may be a substrate or scaffold, for example a biocompatible polymer substrate or scaffold. In one embodiment, the mold may be a non-rigid, flexible or resiliently deformable, polymer scaffold or substrate. The mold may be constructed from a material selected from the group consisting of a gel, agarose, polystyrene, polypropylene, polyethylene, polyethylene terephthalate, polyisoprene, polybutadiene and silicone. The mold may also be a biocompatible polymer selected from the group consisting of matrigel, fibronectin, laminin and collagen. The mold may be 2D or 3D. In particular, the mold may be a 2D or 3D PDMS (polydimethylsiloxane) mold.

In one embodiment, the signal emitting agents may be selected from the group consisting of fluorochromes, fluorescent microspheres, fluorescent-labelled cells, luminescent particles, phosphorescent particles and magnetic particles.

In one embodiment, the culturing step b) of the method as described herein comprises culturing the pre-selected cells in a serum-free medium. The culturing step may be performed for 1 to 10 days, 2 to 10 days, 2 to 9 days, 2 to 8 days, 3 to 8 days or 3 to 7 days.

The cardiac construct may comprise extracellular matrix proteins characteristic of a cardiac construct. The extracellular matrix proteins may comprise various isoforms of laminin, collagen type I, collagen type IV, entactin, proteoglycans including but not limited to heparan sulfate, perlecan and fibronectin.

The contractile cellular construct may be a contractile cellular monolayer construct, a two-dimensional contractile cellular construct or a three-dimensional contractile cellular construct.

In another aspect, the present invention also provides a contractile cellular construct produced by the method as described herein. The cellular construct may be a three-dimensional cardiac construct.

In another aspect, the present invention also provides a method of measuring the contractility of the contractile cellular construct as described herein, comprising: a) measuring the location of the signal emitting agents in the contractile cellular construct, at two or more pre-selected times; and b) determining the temporal change in the location of the signal emitting agents to produce a contraction profile and relaxation profile based upon the measurements of a).

In one embodiment, the measuring step may comprise real-time video recording.

In one embodiment, the determining step may comprise image tracking analysis.

In some embodiments, the contraction profile may comprise at least one contraction parameter selected from the group consisting of contraction pattern, contraction amplitude, contraction time, contraction velocity and acceleration vector.

The relaxation profile may comprise at least one relaxation parameter selected from the group consisting of relaxation pattern, relaxation amplitude, relaxation time, relaxation velocity and acceleration vector.

In another aspect the present invention provides a method for screening one or more agents for modulating the contractility of a contractile cellular construct, comprising: a) contacting the contractile cellular construct as described herein with said one or more agents; b) measuring the location of the signal emitting agents, comprised in the contractile cellular construct, at two or more pre-selected times; c) determining the temporal change in the location of the signal emitting agents in said two or more pre-selected times, to produce a test contraction profile and test relaxation profile based upon the measurements of b); d) comparing the test contraction profile and test relaxation profile of c) with a control contraction profile and control relaxation profile of a contractile cellular construct as described herein that has not been contacted with the one or more agents or has been contacted with the one or more agents at a different concentration to that of step a); wherein a differential profile between the test and control contraction or relaxation profiles demonstrates a modulating activity of said one or more agents on the contractile cellular construct.

In one embodiment, the measuring step may be performed by real-time video recording. In another embodiment, the measuring step may be performed by image tracking analysis.

In another embodiment, the contraction profile comprises at least one contraction parameter selected from the group consisting of contraction pattern, contraction amplitude, contraction time, contraction velocity and acceleration vector. The relaxation profile may comprise at least one relaxation parameter selected from the group consisting of relaxation pattern, relaxation amplitude, relaxation time, relaxation velocity and acceleration vector.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non- limiting examples. In addition, where features or aspects of the invention are described in terms of Markush groups, those skilled in the art will recognize that the invention is also thereby described in terms of any individual member or subgroup of members of the Markush group.

### EXPERIMENTAL SECTION

Materials and Methods

Manufacturing PDMS molds and tissue holders

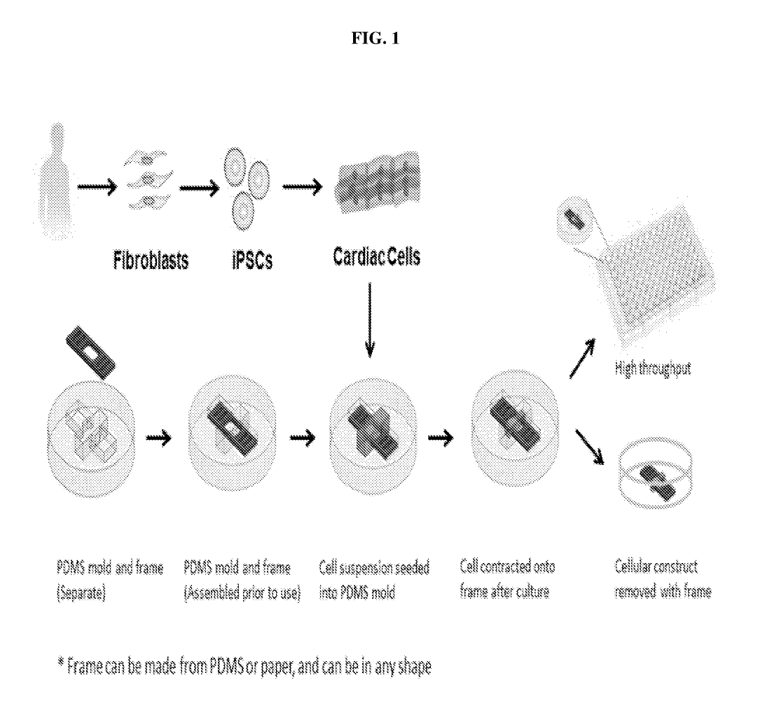

Sylgard 184 silicone elastomer (Dow Corning) was used to prepare silicone molds and tissue holders. The custom-made casting molds were fabricated using 3D printing system (Stratasys, Objet). The PDMS pre-polymer solution containing a mixture of PDMS oligomers and a reticular agent from Sylgard 184 (10:1 mass ratio) was degassed under vacuum conditions before casting. The mixture was poured into the casting molds and cured at 80° C. overnight. Once demoulded, the PDMS substrate was carefully prepared with respect to the dimensions of cell culture wells dimensions. The PDMS substrates were then cleaned with ethanol and air-dried in laminar hood. The silicone molds and tissue holders were designed with dimensions to fit into wells of multi-well culture plates. For 96-well tissue culture plate (NUNC), PDMS mold diameter: 6 mm; Tissue channel: length/width/depth=5 mm×1.5 mm×1.5 mm; tissue holders: length/width=3 mm×2 mm (FIG. 1).

Directed differentiation of hiPSCs into cardiac lineage cells under defined culture conditions

Cardiac differentiation of hiPSCs was carried out under serum-free condition. Typically, the patient derived iPSCs and healthy control iPSCs were plated on Matrigel-coated tissue culture plates in E8 medium to reach near confluence. The cells were washed with PBS, and exposed to medium consisting of RPMI 1640 supplemented with B27 minus insulin (Life Technologies) and CHIR99021 (12 μM, Tocris). After 24-hour treatment, the medium was replaced with RPMI 1640 supplemented with B27 minus insulin. Forty-eight hours later, the medium was changed to RPMI 1640 supplemented with IWP4 (5 μM, Stemgent). After 48-hour treatment, the medium was changed to RPMI 1640 supplemented with B27 minus insulin for two additional days followed by medium replacement to RPMI 1640 supplemented with B27 (Life Technologies) with medium change every two days.

For cardiac differentiation under xeno-free condition, hiPSCs were plated in E8 medium on Laminin521 (BioLamina)-coated culture plates to reach near confluence. The cells were treated with medium consisting of E5 supplemented with CHIR99021 (10 μM, Tocris), TGF β (1 ug/ml, R & D), Y-27632 (5 uM) and 1× concentrated lipid (Life Technologies) for 24 hours. The medium was changed to E5 supplemented with TGFβ (1 ug/ml) and 1× concentrated lipid for two days, then replaced with E5 supplemented with IWP4 (4 uM) and concentrated lipid. After 48-hour treatment, the medium was changed to RPMI 1640 supplemented with B27 Xeno (B27 Supplement CTS, Life Technologies) with medium change every two days.

Generation of 3D cardiac tissues under defined culture condition

To generate 3D cardiac tissues, 12-day differentiated cardiac cells were detached from culture wells using accutase (Stem Cell Technologies), suspended in RPMI-B27medium containing Y-27632 (5 uM) to reach a final concentration of 1.5×10⁸ cells/mL. Casting molds were prepared by placing the tissue holder and PDMS molds in 96-well culture plates. For each tissue, 12 μL cell suspension was loaded into the cell loading channel. For florescence-labeled cardiac tissue, 12 μL cell suspension was mixed briefly with ˜20 FluoSpheres polystyrene microspheres (diameter 15 μm, Life Technologies) and pipetted into cell loading channels. The 96-well plates were placed in a 37° C., 5% CO₂ culture incubator for 2 hours. 0.20 mL of cell culture medium was then added per well for continued culture. After one day of culture, media was changed to RPMI-B27 medium without Y-27632 and changed every day. After 3 days of culture, the cell constructs had formed tissues with sufficient mechanical properties to allow them to retain their integrity upon removal from PDMS molds and during manipulation with forceps.

Seeding cardiomyocytes in 384-well/96-well plates for drug screening

The 12-day differentiated cardiomyocytes were detached using accutase and suspended in RPMI-B27 medium containing Y-27632 (5 uM). The cell suspension was mixed with FluoSpheres polystyrene microspheres, and loaded into matrigel or fibronectin coated 384-well microplate (Greiner) or 96-well tissue culture plates at 0.25˜0.30 *10⁶cells/50˜100 microspheres/cm². After one day of culture, media was changed to RPMI-B27 medium without Y-27632 and changed every day. Cardiomyocyte monolayer was formed after one day, and started to contract synchronously 2 day post-seeding.

Assessment of cardiac tissue contractility

Cardiac tissue contractility was assessed based on the recorded video using IMARIS software. The cardiac tissues were labeled with fluorescence beads, as described above. The video was exported as image stacks for IMARIS tracking analysis. The software can identify the contraction and relaxation events based on the relative positions of the florescence beads during the entire tissue beating sequence. These positions were recorded, and the relevant contractility parameters were calculated from the temporal change of these positions.

Pharmaceutical tests

Toxicity studies were performed using a Zeiss fluorescence microscope, within a closed environment chamber maintaining constant 37° C. temperature and 5% CO2 humidified air for long time lapse imaging of live cells. An experiment design program in Zen software was used to create an automatic measurement program. For chronic drug response, videos were recorded for each well at every 1 h interval.

After 7 days of culture, the 3D cardiac tissues exhibiting good beating activity were subjected to measurement of drug toxicity. The cardiac tissues were equilibrated in RPMI 1640/B27 for one hour and incubated consecutively with increasing concentrations of drugs, including erythromycin, trovafloxacin, ampicillin, rosiglitazone, troglitazone, metformin, chromanol 293B, quinidine sulfate, and E-4031. The videos were recorded at cumulative concentrations 1 hr before measurement, and processed using IMARIS software.

After culture in 384-well microplate for 7 days, the cardiomyocytes were exposed to pentamidine under the following conditions: 1) cumulative concentrations (0.1, 1, 10, 100 μM) for 1 hr; 2) 1, 5, 10 μM for 2 days. The videos were recorded for each well at every 1 hr interval and processed using IMARIS software.

Toxicity assays based on ATP activity

Cardiac spheroids were incubated with various concentrations of compounds dissolved in culture medium for 1 hr, and cell viability was subsequently measured by CellTiter-Glo® 3D cell viability assay (Promega), which determines the number of viable cells in culture based on quantitation of the ATP present. Data is normalized to drug-free controls. Data from the same treatment on 3 occasions were averaged to represent the mean ATP measurement.

Flow cytometry

The differentiated cardiomyocytes were dissociated with Accutase for 6-10 minutes at 37° C., followed by gentle trituration to a single-cell suspension. The cells were processed for staining with anti-cTnT and analyzed with a BD LSR II.

RNA extraction, reverse transcription and polymerase chain reaction

Total RNA was isolated using Trizol reagent (Life Technologies) according to manufacturer's instructions. Before reverse transcription, RNA samples were treated with DNase I (Life Technologies) to remove contaminating genomic DNA. cDNA was synthesized using SuperScript III Reverse Transcriptase and Oligo [dT]₁₈ primers according to the manufacturer's instructions (Life Technologies). PCR was carried out using Taq DNA Polymerase (Life Technologies) with gene specific primers.

Immunocytochemistry

The differentiated cardiomyocytes and tissues were fixed with 4% paraformaldehyde and immunostained with the antibodies as listed below: mouse anti-a-actinin, mouse anti-cTnT, mouse anti-MYH6, mouse anti-fibronectin, rabbit anti-collagen I, mouse anti-collagen IV, rabbit anti-laminin antibody (Abcam) and rabbit Polyclonal Nkx-2.5 (Life Technologies). Appropriate fluorescence (Alexa-Fluor-488/568)-tagged secondary antibodies were used for visualization (molecular probes, Eugene, USA). 4,6-diamidino-2-phenylindole (DAPI) counterstain was used for nuclear staining. The samples were observed under a Zeiss LSM510 laser scanning microscope and photographed and processed with LSM Image Browser software.

Results

Fabrication of PDMS master mold and tissue holder

FIG. 1 shows a schematic depicting the platform for scaffold-free fabrication of 3D cardiac tissue. Three main components are involved in this approach: PDMS master molds, tissue holders and cardiac cells for loading. The PDMS master mold contains a microchannel for cell loading. The size of the mold is based on the desired experimental scale. For example, for a 96-well tissue culture plate, we designed a PDMS mold (6 mm diameter) with a cell loading channel of dimensions 5 mm×1.5 mm×1.5 mm (length/width/depth). Tissue holders, prepared according to the size of the cell loading channels, are made from either nitrocellulose membrane paper or PDMS. Cardiac cells for loading were generated as described below.

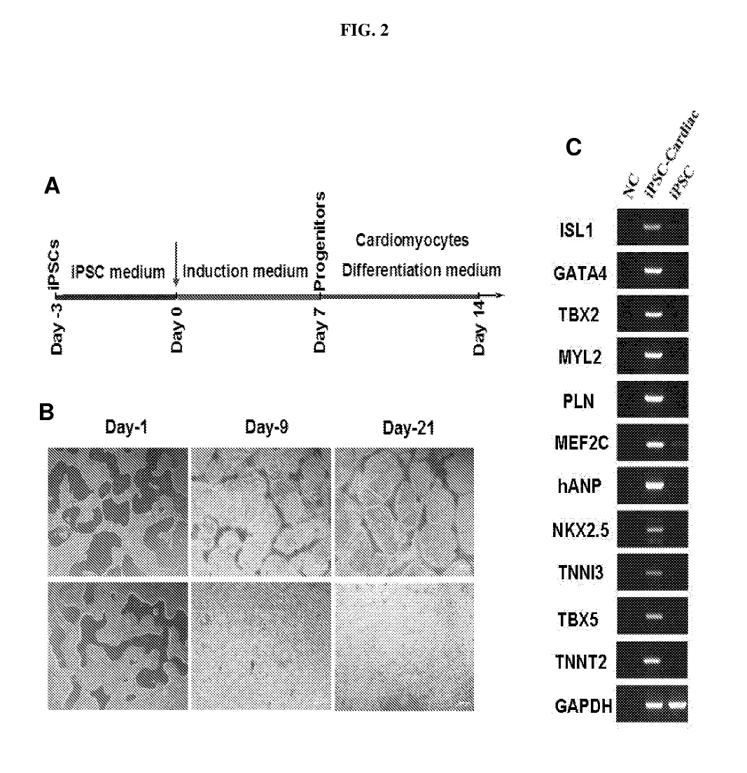

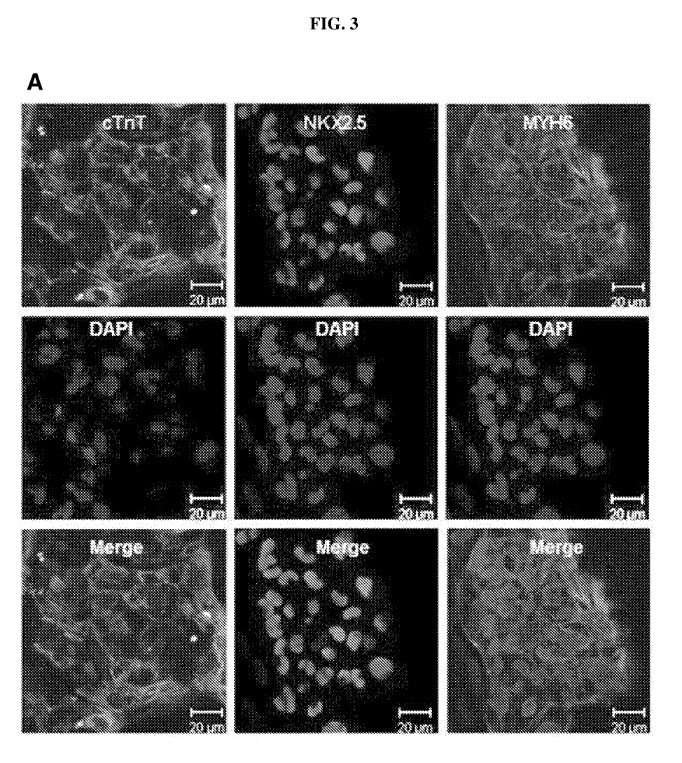

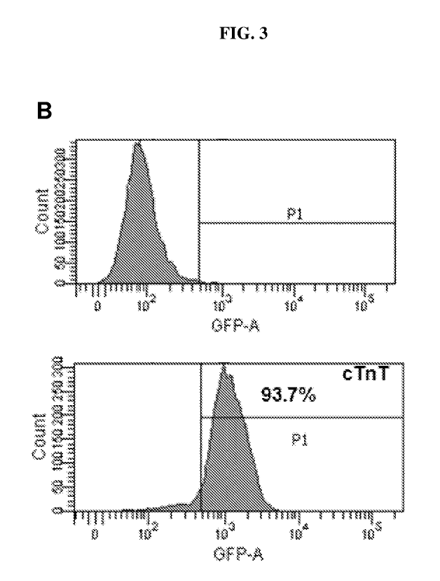

Differentiation of hiPSCs into cardiomyocytes under serum free culture condition The quality of cardiomyocytes is critical for achieving functional beating tissue. In our study, the population for fabricating 3D tissue contained >90% cardiac cells. To achieve this, we differentiated hiPSCs into cardiomyocytes using a small molecule treatment approach. hiPSCs were seeded as single cells and cultured in E8 medium on matrigel-coated plates to reach confluence. Differentiation was induced in RPMI/B27 medium lacking insulin and containing CHIR99021, followed by inhibition of Wnt signaling with IWP4 (FIG. 2). The presence of cardiomyocytes can be easily established by visual observation of spontaneously contracting regions. The first beating cluster of cells can be observed as early as 9 days following initiation of cardiac differentiation. Robust spontaneous contraction occurs by day 12. At day 14, PCR analysis showed the expression of cardiac genes in these differentiated cells. Immunostaining demonstrated that the cells showed positive staining for distinct cardiomyocyte markers including MYH6, cTnT and NKX2.5. Flow cytometry with antibody against cTnT revealed the percentage of cardiomyocytes in the differentiated population was greater than 90% (FIG. 3).

Fabrication of 3D cardiac tissues under defined culture conditions

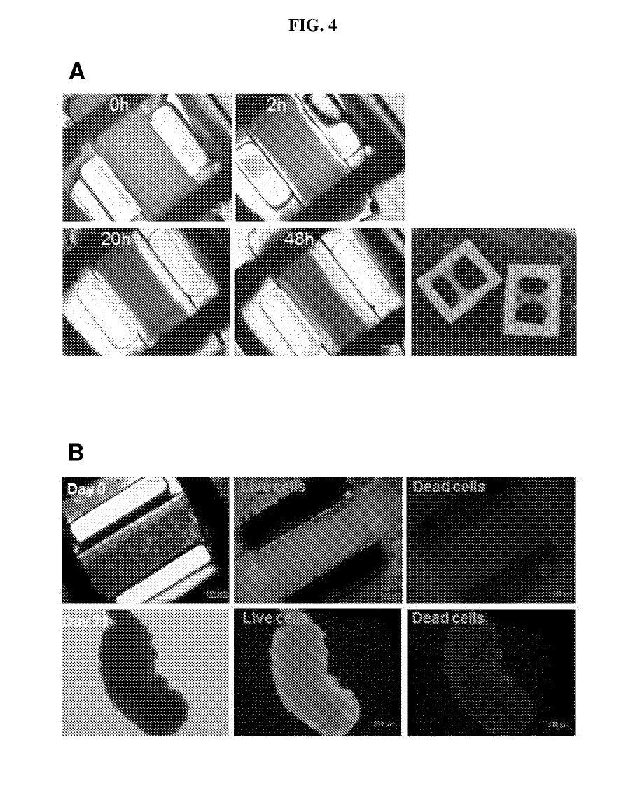

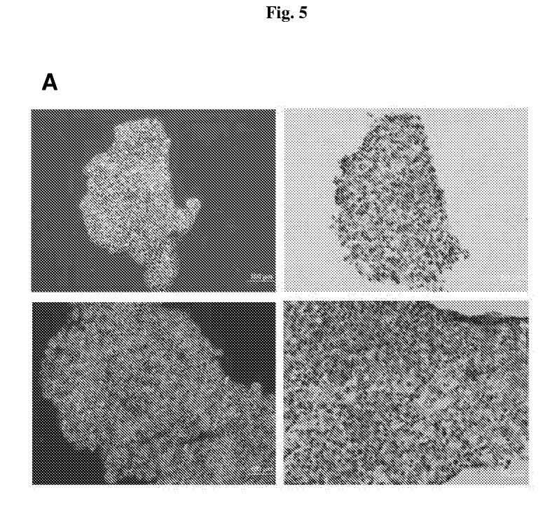

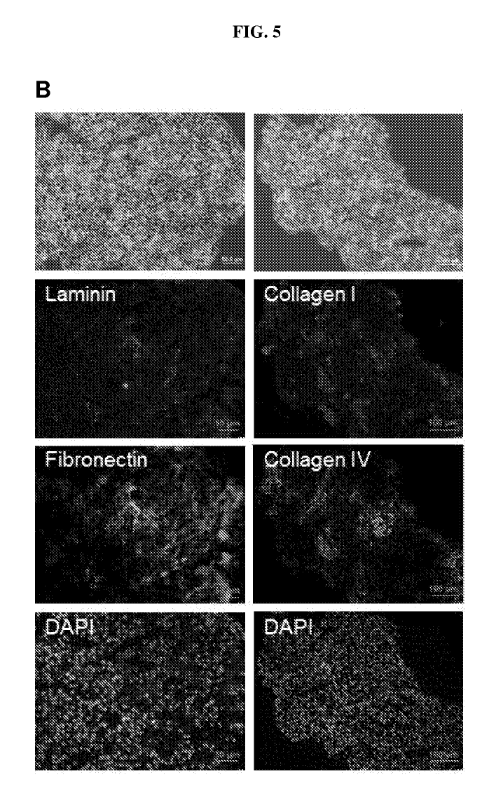

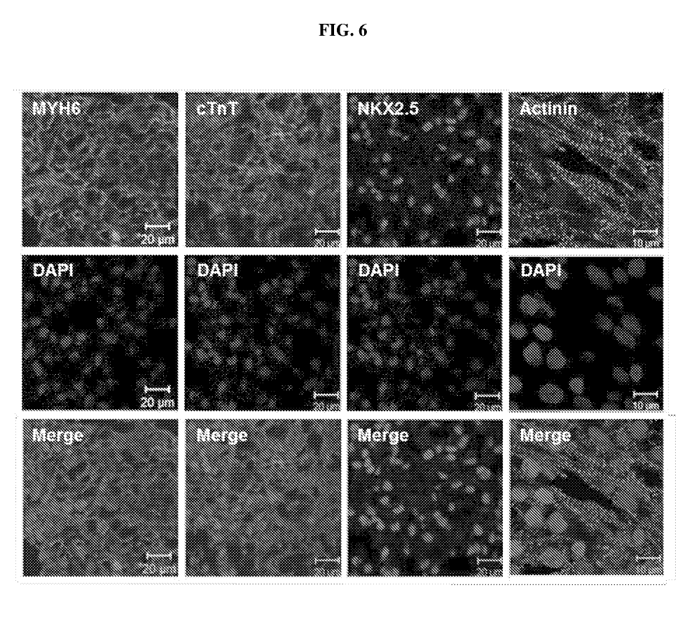

The 12-day differentiated cardiomyocytes were detached and loaded into spatially defined PDMS molds under defined serum-free conditions. Molds were fully immersed in culture medium and the culture was maintained in a 37° C., 5% CO₂ humidified incubator. Over the course of 48 hours, the loaded cells started to aggregate into rod-shaped tissue constructs and exhibited progressive lateral and longitudinal condensation (FIG. 4). Spontaneous contraction of single cells was seen after 1-2 days, and coordinated contraction of entire constructs was observed after 2-3 days, remaining stable for at least 2 months. By day 3 in culture, the cell constructs had formed tissues with sufficient mechanical properties, which allowed them to be removed from the PDMS molds and manipulated with forceps without compromising their structural integrity. Live/dead staining assay for 21-day tissue revealed strong green fluorescence in tissue constructs, suggesting high cell viability in the 3D tissue structure. Light microscopy revealed a reproducible, uniform pattern of cellular distribution. Hematoxylin/eosin staining revealed a dense, well-developed cellular network of heart muscle tissue. Uniform cell distribution with continuous cellular cover was observed throughout sections of the tissue constructs, further confirming a high degree of cell survival. No necrotic region was observable within the 3D tissue construct. Immunostaining against extracellular matrix protein antibodies demonstrated the presence of laminin, type I and IV collagen, and fibronectin in 3D cardiac tissue (FIG. 5), suggesting that the cells synthesized robust ECM rapidly to form self-supporting 3D tissue constructs. Further characterization by immunohistochemistry staining revealed that more than 90% of cells showed the typical marker spectrum of cardiomyocytes: MYH6, α-sarcomeric actin, NKX2.5 and cardiac troponin T (FIG. 6).

Fabrication of fluorescence labelled cardiac tissues for high throughput screening assays

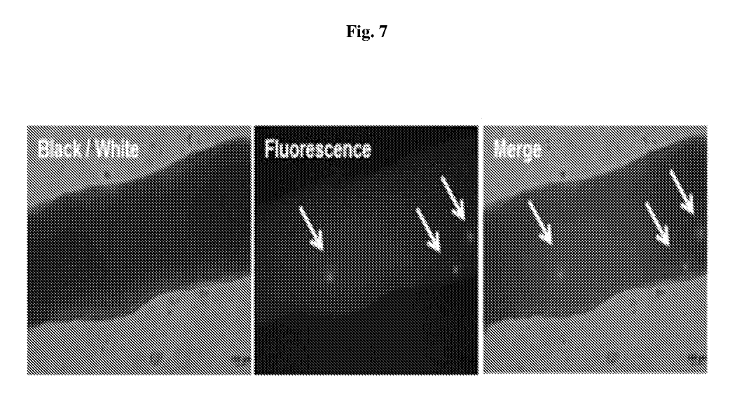

One of the applications for the engineered cardiac tissues is for in vitro toxicity assays. In order to adapt this 3D model to a high throughput screening assay, fluorescence labels were incorporated into the cardiac tissues to enable real time monitoring of cardiac contractile motion. The fluorescence labels which can be used include fluorescent microspheres or cells. After optimization, it was found that microspheres of diameter comparable to that of the cell have negligible effects on tissue structure and contracting function, and also allows monitoring of the beating pattern by automated video-optical recording. The procedure is described below using FluoSpheres polystyrene microspheres (diameter: 15 μm) as an example. For 3D cardiac tissues: 12-day differentiated cardiomyocytes were detached, mixed with fluorescence microspheres (˜20 beads/tissue), and loaded into spatially defined PDMS molds under defined serum-free conditions. After three days, synchronized contractile 3D cardiac tissues were formed, and were either left inside or removed from the PDMS molds for continued culture (FIG. 7).

Automatic analysis of contractile motion of 3D fluorescence labelled cardiac tissues

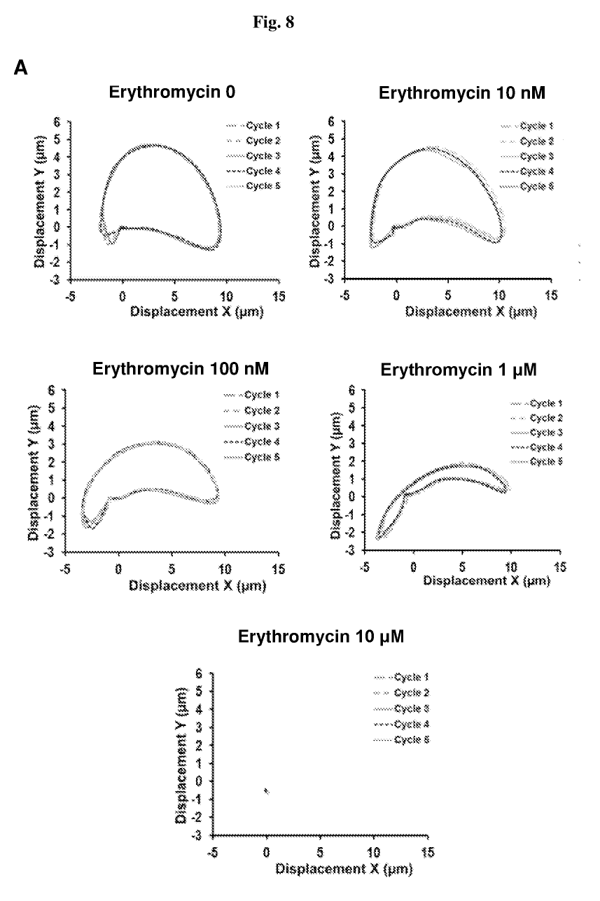

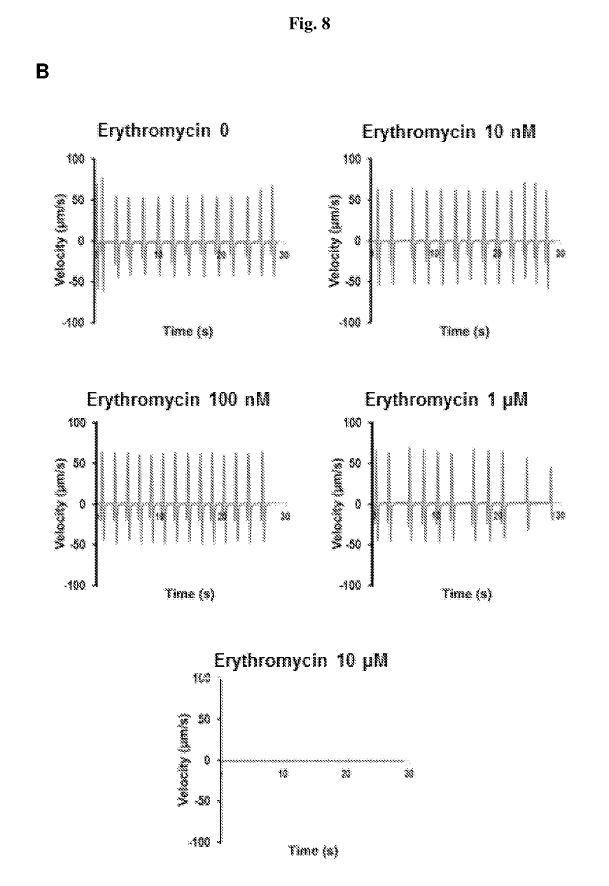

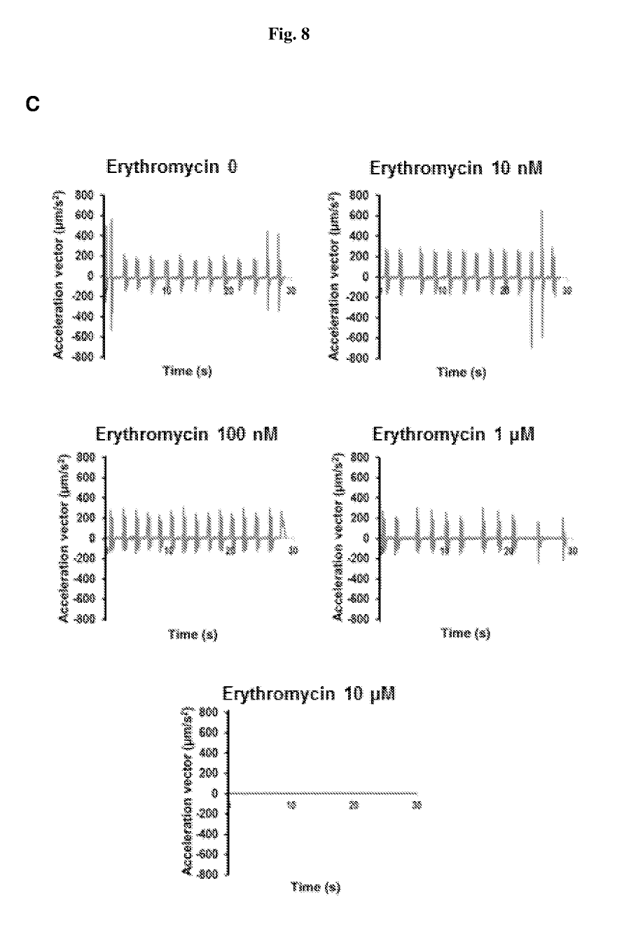

A methodology has also been developed to analyse the contractile motion of the 3D fluorescence labelled cardiac tissues to enable evaluation of cell tissue responses in screening assays. Real-time videos were taken of the cardiac tissues and IMARIS software was used to track and analyse the real-time positions of the fluorescent label. This software both enables processing of the data and provides selectable outputs for analysis. In this way, contraction and relaxation events can be identified based on the detection of florescence signals from the beads during the entire tissue beating sequence. Subsequently, the relevant contractility parameters can be calculated from the temporal change of these positions. Therefore, with this methodology, we are able to obtain quantitative information of 3D cardiac tissue contraction profile, including tissue contraction pattern, amplitude, time, velocity and acceleration vector, which is important in evaluating cardiac functional response in screening assays (as shown in FIG. 8). Automated analysis further facilitates the assays to be performed at high throughput.

Pharmacological study using 3D fluorescence labelled cardiac tissues

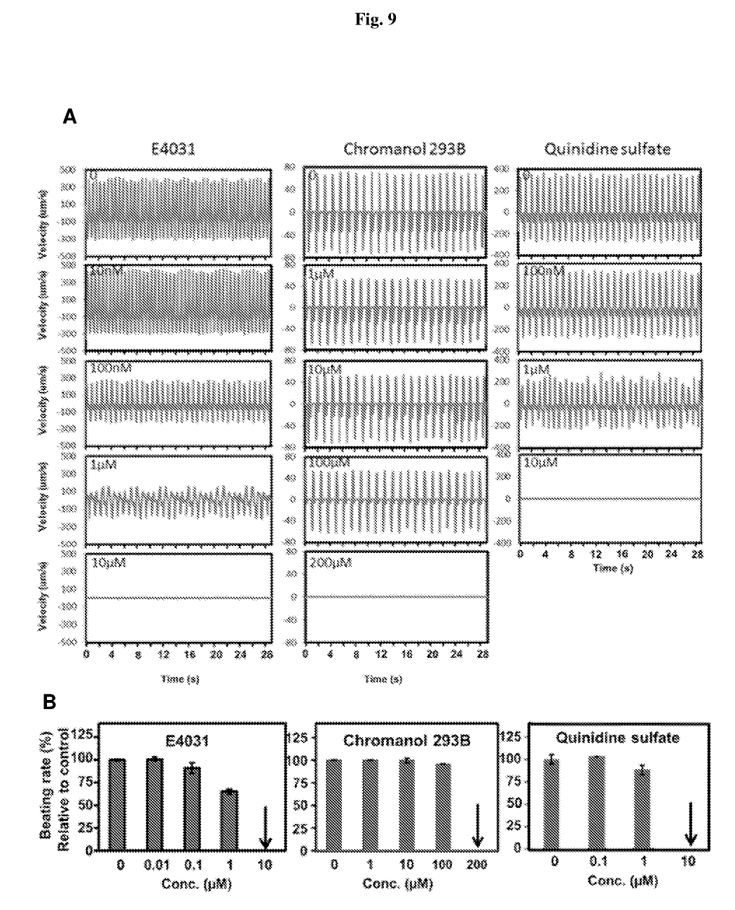

The suitability of the formed 3D cardiac tissue models for pharmacological screening was validated. One-week fluorescence labelled contractile cardiac tissues were subjected to measurement of drug toxicity. The cardiac tissues were exposed to several cardioactive drugs known to block Iₖᵣ, prolong the QT interval, or induce Torsades de Pointes including E-4031, chromanol 293B, erythromycin and quinidine. Cell tissue responses were evaluated with real-time video recording and IMARIS software. Based on the fluorescence video recording, IMARIS software gives quantitative information including tissue contraction pattern, time, velocity and acceleration vector. Drugs exerted a decrease in contraction frequency of the cardiomyocytes and abnormal contraction patterns. Complete arrest of contraction was observed at 10 μM for erythromycin, 200 μM for chromanol 293B, 10 μM for E4031 and 10 μM for quinidine respectively (FIGS. 8 and 9). After washing with Dulbecco's Phosphate-Buffered Saline and overnight incubation in fresh RPMI/B27 medium, cardiac tissues resumed spontaneous contraction by the next day and could be used for a second round of toxicity testing, with consistent results.

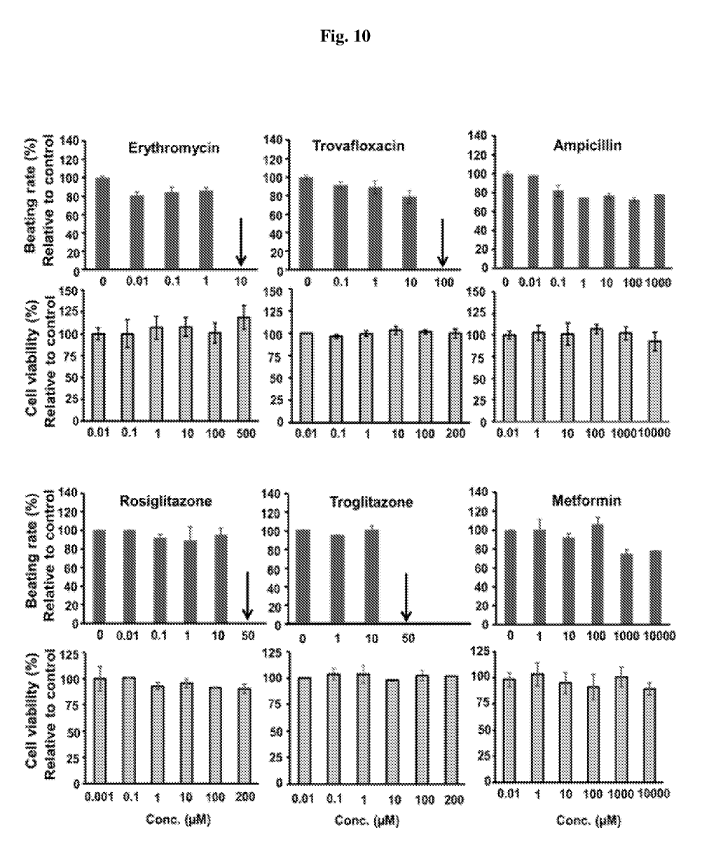

Next, the toxicity effect of a panel of clinical drugs, including antibiotics and antidiabetics, was tested by exposing the 3D cardiac tissues to increasing concentrations of these drugs. For comparison purposes, the toxicity effect of these drugs were studied using ATP-based cell viability assays in parallel (FIG. 10). Rosiglitazone (Avandia), troglitazone (Rezulin) and metformin are a group of antidiabetic drugs. Rosiglitazone and troglitazone have recently been reported to cause an increase in cardiovascular risk in Type 2 diabetic patients. Interestingly, ATP-based cell viability analyses revealed negligible toxicity of rosiglitazone or troglitazone on cardiomyocytes at concentrations up to 200 μM. However, when treated with increasing concentrations of these drugs, the 3D cardiac tissues stopped contraction at 50 μM of both drugs. Treatment of the 3D cardiac tissues with increasing concentrations of metformin showed less pronounced changes in beating rates, implying negligible cardiotoxicity of this drug in the measured concentration range. Ampicillin, erythromycin, and trovafloxacin (Trovan) belong to the antibiotic class of compounds. Cell viability analyses revealed that no significant toxicity was detected at concentrations of up to 500 μM, 200 μM, and 10000 μM of erythromycin, trovafloxacin and ampicillin, respectively. In contrast, the cardiotoxic effect of erythromycin and trovafloxacin was observed at 10 μM and 100 μM, where the contractions stopped in the 3D functional models of this study. However, negligible beating rate changes were observed in the presence of ampicillin, even at concentrations up to 1 mM, suggesting a safe cardiotoxicity profile of this drug within the tested concentration range. Taken together, these results indicate the importance of using a cardiac functional assay for precise prediction of cardiac safety in drug development

Pharmacological study using 2D fluorescence labelled cardiac tissues

The fluorescent labelling technique was extended to 2D high throughput format. Although advances in high throughput systems for drug screening have been made, current 2D cardiac models adaptable to high-throughput array formats, for example, 384-well plate are limited. Furthermore, most 2D models (using Ca channel sensitive dye or genetic modified cardiomyocytes) are not suitable for chronic toxicity evaluation, which play an important role in long-term patient treatment outcomes. Combined with the fluorescent labelling technique disclosed herein, the 2D fluorescence labelled cardiac model has several advantages: 1) Easily adaptable to 384-well plate or even smaller microplates. 2) Any type of cardiomyocyte can be used, including non- genetic/genetic modified cells, 3) Both short-term (mins, hrs) and long-term (days, weeks) drug effects can be evaluated.

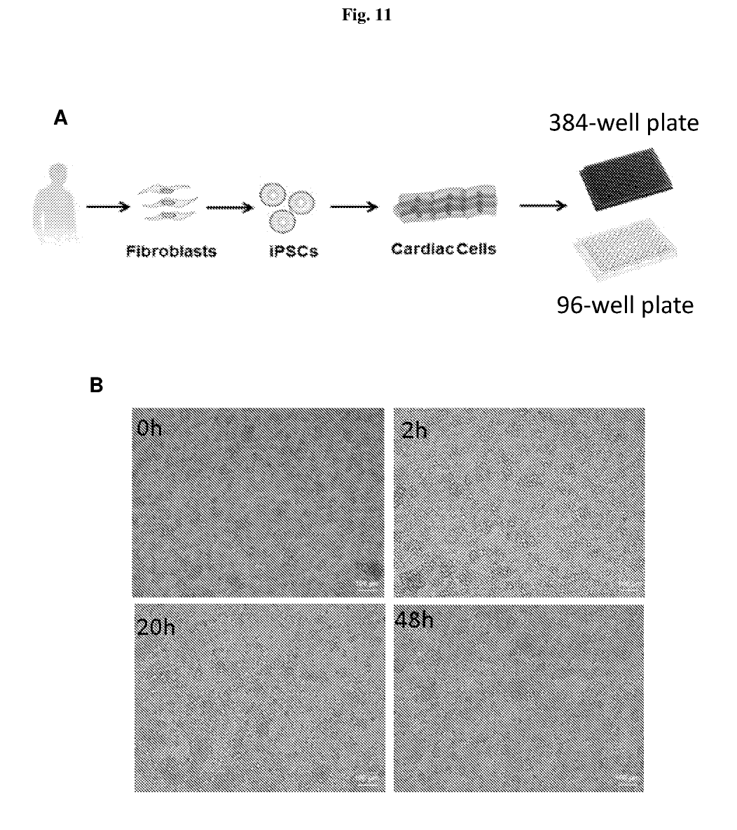

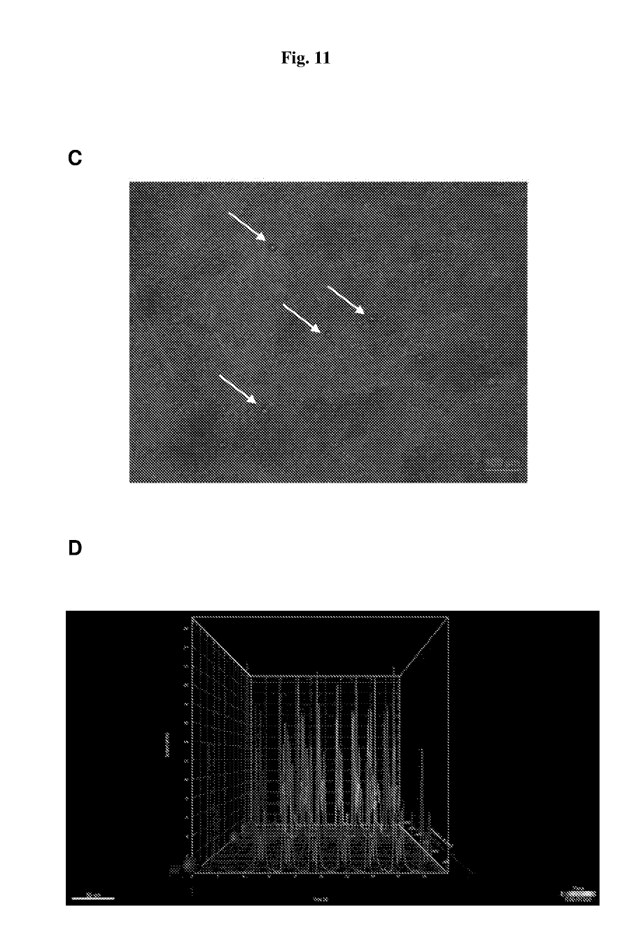

FIG. 11 shows the preparation of 2D cardiac model for high throughput drug screening. The 12-day differentiated cardiomyocytes were detached, mixed with fluorescence microspheres, and loaded into matrigel or fibronectin coated 384-well microplate (or 96-well tissue culture plate) at 0.25˜0.30*10⁶cells/50˜100 microspheres/cm². Cardiomyocyte monolayer was formed after one day, and started to contract synchronously 2 day post-seeding (FIG. 11 B-D). Cell seeding density is important in the 2D cardiac functional assay, which requires formation of cell monolayer and synchronous contraction.

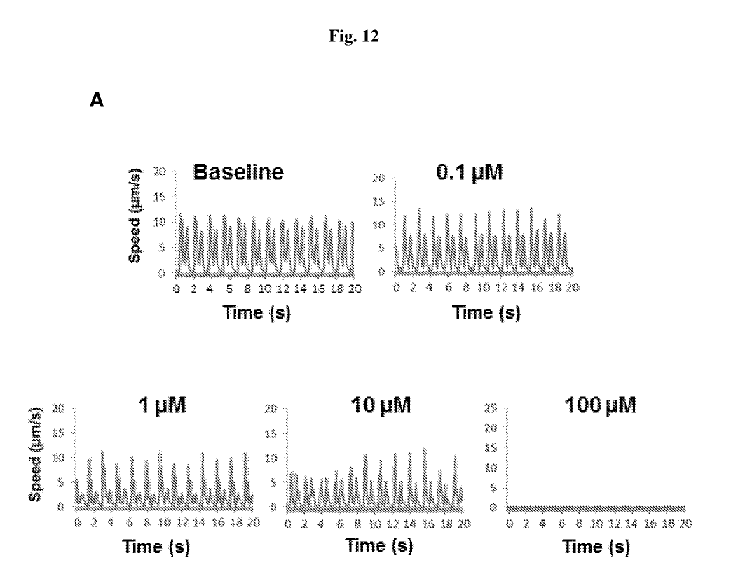

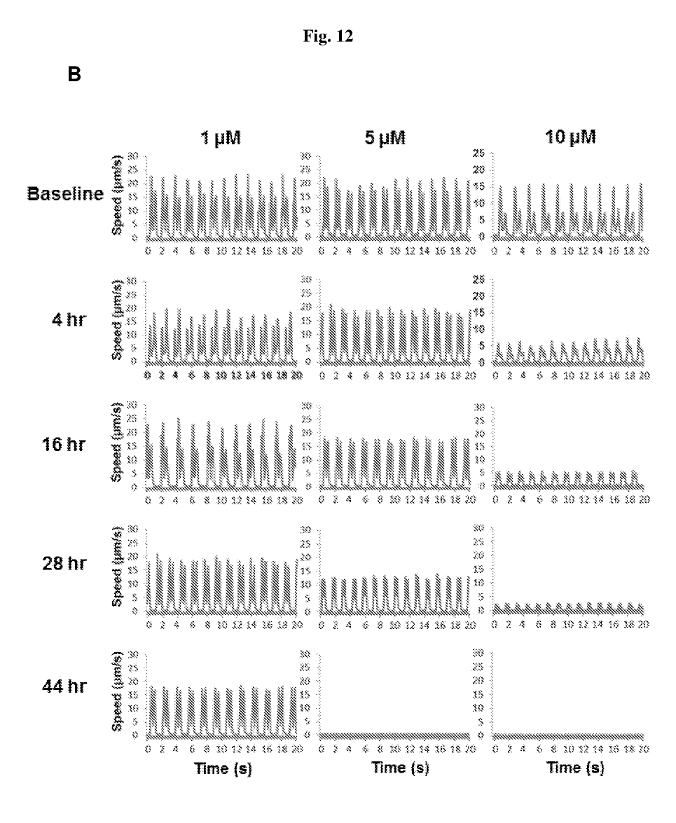

Pentamidine toxicity effect was evaluated using the 2D fluorescence-labelled cardiomyocyte monolayer. Pentamidine is an antiprotozoal agent, which is used in the treatment of Pneumocystis carinii pneumonia. However, therapy with pentamidine is often accompanied by prolongation of the QT interval. In this study, treatment of the 2D cardiac monolayer with increasing concentrations of pentamidine for 1 hr showed pronounced changes in contraction speed (FIG. 12A) at 10 μM, and a complete arrest of beating was observed at 100 μM. However, when cardiomyocytes were exposed to pentamidine at 1 μM, 5 μM and 10 μM for 2 days, significant reduced beating speed was observed at 4 hr (10 μM pentamidine) and 28 hr (5 μM pentamidine), and total arrest of beating at 30 hr (10 μM pentamidine) and 44 hr (5 μM pentamidine). No drug-induced changes were observed until 2-day postdose for 1 μM pentamidine (FIG. 12B).

Potential applications

This technique describes a novel protocol to fabricate 3D functional cardiac tissues under defined conditions. This approach allows uniform cell inclusion within constructs, creating 3D geometrically controlled microenvironments favourable for direct cell-cell self-organization of appropriate 3D ECM assembly with complex cell-matrix and cell-cell interactions that mimic functional properties of the corresponding tissue. Thus, this approach provides a simple model for recapitulating and better understanding physiologically relevant issues at native heart tissue level.

Some potential biomedical applications are listed as follows:

Cell-Based drug testing and high-throughput screening

Compared to other scaffold- or ECM-based 3D cell models, the present model recapitulates the in vivo cellular environment, better mimicking the native heart tissue architecture. As demonstrated in the experiments, the system allows simultaneous monitoring of cardiac tissue force generation, while reporting rapid changes in tissue contraction in response to drug stimuli. With its highly customizable design and fluorescence labelling method, this platform represents a unique approach to quantify the impact of drug on function of 3D cardiac tissues, thus showing great promise to be used for high-throughput, low-cost screening assays for pharmaceutical drug development.

Cardiovascular disease model

This technique allows for the generation of cardiac tissues from patients in the context of particular genetic identity, including individuals with sporadic forms of disease. These 3D cardiac tissue models will be ideally suited to test disease progression.

Basic study

By tailoring the mechanical modulus of the tissue holders, our approach can provide a simplified model with which to investigate directly the effect of different mechanical stresses on cardiomyocyte alignment and growth, and measuring the functional consequences of these interventions. This could also provide a platform to study the mechanical effect of infarct cardiac muscle on the surrounding healthy contractile tissues.

Cell-Based therapy

The 3D cardiac tissues generated from patient iPSCs under defined xeno-free conditions will have great potential for clinical cell-based therapy.

1. A method for producing a contractile cellular construct, comprising the steps of:
a) seeding pre-selected cells onto a silicone mold, wherein the pre-selected cells comprise signal emitting particles; and
b) culturing the pre-selected cells to produce the contractile cellular construct.

2. The method of claim 1, wherein prior to step a) the method further comprises: inducing a pluripotent stem cell into a pre-determined lineage of the pre-selected cells; isolating the induced pre-selected cells; and contacting the isolated pre-selected cells with signal emitting particles to produce pre-selected cells comprising signal emitting particles; optionally wherein the pluripotent stem cell is a human induced pluripotent stem cell (hiPSC); optionally wherein the hiPSC is derived from a biological sample.

3. (canceled)

4. (canceled)

5. The method of claim 1, where the pre-selected cells are derived from a biological sample; optionally wherein prior to step a) the method further comprises: isolating the pre-selected cells from a biological sample; and contacting the isolated pre-selected cells with signal emitting particles to produce pre-selected cells comprising signal emitting particles.

6. (canceled)

7. The method of claim 1, wherein the contractile cellular construct is a muscle construct; optionally wherein the muscle construct is selected from the group consisting of skeletal muscle construct, cardiac muscle construct and smooth muscle construct.

8. (canceled)

9. The method of claim 1, wherein the contractile cellular construct is a cardiac muscle construct, and the pre-selected cells are cardiac cells; optionally wherein the cardiac cells comprise one or more mammalian cells selected from the group consisting of cardiomyocytes, endocardial cells, cardiac adrenergic cells, endothelial cells, neuromuscular cells and cardiac fibroblasts; optionally wherein the cardiomyocytes comprise one or more of ventricular cardiomyocytes, atrial cardiomyocytes and nodal cardiomyocytes; optionally wherein the cardiac cells comprise cardiomyocytes expressing one or more markers selected from the group consisting of MYH6, α-sarcomeric actin, cTnT, Connexin 43, GATA4, Tbx5, MEF2c, sarcomeric MHC, sarcomeric actinin, Cardiac troponin I, atrial natriuretic peptide, Smooth muscle α-actin, desmin and NKX2.5.

10-12. (canceled)

13. The method of claim 9, wherein at least 70%, 80% or 90% of the cardiac cells are cardiomyocytes.

14. (canceled)

15. The method of claim 1, wherein the mold is a spatially defined PDMS mold.

16. The method of claim 1, wherein the signal emitting particles are selected from the group consisting of fluorescent microspheres and magnetic particles.

17. The method of claim 1, wherein the culturing step b) comprises culturing the pre-selected cells in a serum-free medium; optionally wherein the culturing step is performed for 1 to 10 days, 2 to 10 days, 2 to 9 days, 2 to 8 days, 3 to 8 days or 3 to 7 days.

18. (canceled)

19. The method of claim 9, wherein the cardiac construct comprises extracellular matrix proteins characteristic of a cardiac construct optionally wherein the extracellular matrix proteins comprises laminin, collagen type I, collagen type IV and fibronectin.

20. (canceled)

21. The method of claim 1, wherein the contractile cellular construct is a contractile cellular monolayer construct, a two-dimensional contractile cellular construct or a three-dimensional contractile cellular construct.

22. A contractile cellular construct produced by the method of claim 1.

23. The contractile cellular construct of claim 22, wherein the cellular construct is a three-dimensional cardiac construct.

24. A method of measuring the contractility of the contractile cellular construct of claim 22, comprising:
a) measuring the location of the signal emitting particles in the contractile cellular construct, at two or more pre-selected times; and
b) determining the temporal change in the location of the signal emitting particles to produce a contraction profile and relaxation profile based upon the measurements of a), wherein the contraction profile comprises at least one contraction parameter selected from the group consisting of contraction pattern, contraction amplitude, contraction time, contraction velocity and acceleration vector.

25. The method of claim 24, wherein the measuring step comprises real-time video recording; optionally wherein the determining step comprises image tracking analysis; optionally wherein the relaxation profile comprises at least one relaxation parameter selected from the group consisting of relaxation pattern, relaxation amplitude, relaxation time, relaxation velocity and acceleration vector.

26-28. (canceled)

29. A method for screening one or more agents for modulating the contractility of a contractile cellular construct, comprising:
a) contacting the contractile cellular construct of claim 22 with said one or more agents;
b) measuring the location of the signal emitting particles, comprised in the contractile cellular construct, at two or more pre-selected times;
c) determining the temporal change in the location of the signal emitting particles in said two or more pre-selected times, to produce a test contraction profile and test relaxation profile based upon the measurements of b);
d) comparing the test contraction profile and test relaxation profile of c) with a control contraction profile and control relaxation profile of a contractile cellular construct of claim 22 that has not been contacted with the one or more agents or has been contacted with the one or more agents at a different concentration to that of step a);
wherein a differential profile between the test and control contraction or relaxation profiles demonstrates a modulating activity of said one or more agents on the contractile cellular construct, wherein the contraction profile comprises at least one contraction parameter selected from the group consisting of contraction pattern, contraction amplitude, contraction time, contraction velocity and acceleration vector.

30. The method of claim 29, wherein the measuring step is performed by real-time video recording; optionally wherein the measuring step is performed by image tracking analysis; optionally wherein the relaxation profile comprises at least one relaxation parameter selected from the group consisting of relaxation pattern, relaxation amplitude, relaxation time, relaxation velocity and acceleration vector.

31-33. (canceled)
